(19) United States
(12) Patent Application Publication
(10) Pub. No.: US 2022/0071603 A1
(43) Pub. Date: Mar. 10, 2022

(54) Device and Method for Prediction and Characterization of a Conception Cycle

(71) Applicant: Lady Technologies, Inc., San Francisco, CA (US)

(72) Inventors: Kristina Cahojova, San Francisco, CA (US); Scott Shwarts, San Francisco, CA (US)

(21) Appl. No.: 17/530,788

(22) Filed: Nov. 19, 2021

Related U.S. Application Data
(63) Continuation-in-part of application No. 16/491,926, filed on Sep. 6, 2019.

(51) Int. Cl. A61B 10/00; G16H 10/40; A61B 5/00

(52) U.S. Cl. CPC A61B 10/0012; G16H 10/40; A61B 5/0022; A61B 5/743; A61B 2010/0032; A61B 5/4331; A61B 2010/0016; A61B 2010/0019; A61B 5/4337

(57) ABSTRACT

The device and method for calculating and displaying to a user a predicted fertility window, start and end date, and conception planning guide for the user is presented. The device and method include a vaginal sensor device having a plurality of sensors to collect measurements related to vaginal and cervical parameters that are important to the conception cycle. The device and method are customized for an individual user through the input by a user of menstrual cycle timing and duration, ovulation data, temperature data, and other data that are personal to the user. The system utilizes all collected sensor measurements, input user data, and historical data to provide a predicted fertility window, including predicted start and end dates, for the user.

### COPYRIGHT NOTICE

A portion of the disclosure of this patent document contains material which is subject to copyright protection. The copyright owner has no objection to the facsimile reproduction of the patent document or the patent disclosure, as it appears in the

Patent and Trademark Office patent file or records, but otherwise reserves all copyright rights whatsoever.

### CROSS-REFERENCE TO RELATED APPLICATION

This application is a continuation-in-part of U.S. application Ser. No. 16/491,926, filed Sep. 6, 2019, and entitled “Vaginal Device and Method for Measuring Fertility-Related Parameter”.

### BACKGROUND

When trying to conceive, most women want to determine the simplest and most accurate way to confirm that they ovulated and how to best time their intercourse to increase their chance of conceiving. Ovulation can be detected in a variety of ways. Common at-home methods include tracking basal body temperature, also known as BBT, and measuring Luteinizing Hormone (LH) levels. Both techniques have benefits and limitations.

Measuring LH (Luteinizing Hormone) levels (sometimes known as an Ovulation Prediction Kit or OPK) can be helpful in determining if you are having a surge of LH. When LH is high it signals the ovary to release an egg and it can be estimated that you will ovulate within the next 12 to 36 hours. However, it is possible to have a positive LH test without ovulating or ovulate without detecting a positive

LH test. If waiting to time intercourse to a positive LH, many couples might miss crucial days of the fertile window.

Charting Basal Body Temperature (BBT) can confirm ovulation by observing an upward and sustained temperature shift. However, numerous factors may impact BBT including temperature recording method, sleep quality, alcohol consumption, and illness. Physicians can confirm ovulation by performing a transvaginal ultrasound to observe the “ring of fire” of the corpus luteum. Alternatively, doctors may complete an endometrial biopsy or a blood test to measure your progesterone level. Other methods can be used at home to confirm healthy ovulation.

While these methods are beneficial for determining if ovulation has occurred, they do not help identify the fertile window to assist in conception planning.

### BRIEF DESCRIPTION OF THE DRAWINGS

Certain illustrative embodiments illustrating organization and method of operation, together with objects and advantages may be best understood by reference to the detailed description that follows taken in conjunction with the accompanying drawings in which:

FIG. 1 is a cutaway view of the vaginal sensor device consistent with certain embodiments of the present invention.

FIG. 2 is a cutaway schematic view of the vaginal sensor device consistent with certain embodiments of the present invention.

FIG. 3 is a cutaway view of the vaginal sensor device inserted into the vagina of a human female body consistent with certain embodiments of the present invention.

FIG. 4 is a side view of the vaginal sensor device having a tail in usable configuration consistent with certain embodiments of the present invention illustrating the nozzle.

FIG. 5 is a detail view of the vaginal sensor device and recharging cradle consistent with certain embodiments of the present invention.

FIG. 6 is a detail view of the voltage over time graph created during measurement activities of the vaginal sensor device consistent with certain embodiments of the present invention.

FIG. 7 is a graph of cervical impedance vs. a user ovulation cycle collected by the vaginal sensor device consistent with certain embodiments of the present invention.

FIG. 8 is an exemplary screenshot of a home page view of an operation application configured to be used with the vaginal sensor device consistent with certain embodiments of the present invention.

FIG. 9 is an in-use view of the chart page of the operation application in use with the vaginal sensor device consistent with certain embodiments of the present invention.

FIG. 10 is an in-use view of the calendar page of the operation application in use with the vaginal sensor device consistent with certain embodiments of the present invention.

FIG. 11 is an in-use view of the data addition page of the operation application in use with the vaginal sensor device consistent with certain embodiments of the present invention.

FIG. 12 is a flow diagram for the use of the vaginal sensor device to predict and characterize a user fertility window consistent with certain embodiments of the present invention.

### DETAILED DESCRIPTION

While this invention is susceptible of embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings.

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically. The terms ‘activation’ and ‘action of the user’, as used herein, may refer to application of the dispensing force to the actuation button.

Reference throughout this document to “one embodiment”, “certain embodiments”, “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

Timing intimacy with ovulation can be challenging. Trying to conceive only during ovulation, there is typically only a 12 to 24-hour window to be intimate before the egg dies. Depending on what method chosen to confirm ovulation, it could be too late for conception to occur. In a non-limiting example, if tracking Basal Body Temperature to confirm ovulation, a user could have ovulated shortly after waking up one day and not get a temperature rise until the following day. This would mean that an egg released during the current ovulation cycle is unlikely to be viable by the time the user may have intercourse. In another non-limiting example, if using LH tests, the user could miss her ovulation by getting negative LH tests or testing on the wrong days.

Women typically have the most fertile cervical fluid 2-3 days before ovulation. Intercourse on these days tend to offer the best chances for conception. Within the fertility cycle, increasing estrogen causes the electrolyte levels in cervical fluid to change. The vaginal sensor device uses electrical impedance to measure the electrolyte levels of a user when the vaginal sensor device is inserted into the vagina of the user.

Each day's readings may be sent to the cloud via a Near Field Communication protocol such as, in a non-limiting example, a bluetooth connected application running on the user's mobile device. A program in the cloud analyzes the user's past cycles and current cycle looking for a specific pattern in the readings that occurs within a certain window of time where the certain window of time is characterized by the day of ovulation of the user. That pattern is then used to identify a fertile window—a multiple day period within the cycle where a user is most likely to have a successful conception and characterized by impedance readings for the cervical mucus diminishing. The reduction in impedance of the cervical mucus represents the best opportunity for sperm to survive in the uterus long enough to come into contact with one or more eggs that are released during the ovulation event. That window is displayed on the user's smartphone allowing them to take action by planning intercourse during the fertile window and thus increase the chances of conception. Other information is collected by the application and can be used to improve the prediction.

The cervical fluid's impedance is measured by charging one or more electrodes with a specific frequency, low voltage signal and then sensing the voltage and frequency of the signal received on one or more other electrodes. The process is then reversed with the opposite electrodes. By varying the frequency used, the composition of the cervical fluid, the impedance of the cervical mucus, as well as certain chemical attributes (such a pH) can be determined. Other sensing such as temperature can be used to further characterize the fluid composition.

In order to create an actionable fertile window for the user, the cloud process analyzes the impedance readings for the cervical mucus collected by the device. Both positive and negative impedance is measured which is then averaged over time to derive an impedance value for this session. The process looks for the low impedance point occurring within the expected candidate fertile window, where the candidate fertile window may be determined from the user's past ovulation cycles. This candidate is created by finding the low median point from previous cycles. In this way, previous cycles inform future cycle predictions. Within the current cycle, once the user enters a luteal phase, the fertile window from the current cycle can be confirmed by examining the waveform looking for the low point followed by an increase in the impedance value of the cervical mucus. In this embodiment, changes in the cervical fluid detected by the vaginal sensor device are the most accurate way to assess real-time fertility.

Working with the user's unique cycle trends as input by a user, the vaginal sensor device and system analyses collected sensor readings and other data to predict and highlight a 5-day fertile window based around the ovulation cycle. As estrogen rises, the cervix secretes a highly fertile cervical mucus that can nourish and house the sperm for up to 5 days until ovulation can occur. Since the cervical mucus can nourish and support sperm on their journey to the egg, couples actually have the best chance of conceiving from intercourse 2 to 3 days before ovulation. The vaginal sensor device can help users recognize these optimal days as their daily readings descend into the fertile valley, as depicted in FIG. 7.

In an embodiment, the vaginal sensor device may also vary the frequency of the signal being fed to the electrodes within the body of the vaginal sensor device. The sensor readings that are collected during the frequency variation operation of the vaginal sensor device are transmitted to a cloud-based data processor for analysis. The vaginal sensor device may also contain a vibration motor within the device to provide a haptic signal to the user that the collection of measurements from the sensors has begun, that measurements have completed, or that an error has occurred. The haptic signal provided for each of these actions is distinctive and different for each type of activity such that the user may distinguish what action has been taken by the particular haptic signal that is transmitted to the user. In an embodiment, the vibration motor may be active to assist in the performance of kegel exercises. The distinctive vibration signals (haptic signals) provide an ability for the user to indicate the start and end points of kegel exercises, as well as then an error has occurred in the use of the vaginal sensor device.

The analysis of this data is used to create both the duration of a fertile window and the starting and ending date for the fertile window for the upcoming menstruation cycle as customized for each user. The knowledge of this predicted fertile window duration and start and end date effect the user's behavior in terms of the timing of conception providing the ability of the user and their partner to engage in intimate relations during the best, most beneficial period for conception for the particular user's monthly cycle.

In an embodiment, additional conditions and information may be provided to the user as determined by the cloud-based analysis of the data collected from the vaginal sensor device. Conditions such as infection and early signs of cervical health issues may be determined from the analysis of the timing of the user's cycle and analysis of sensor readings from the cervical mucus, cervical fluid, temperature information, and other information that is both collected and entered by the user. Additionally, the information about the predicted fertile window for the upcoming cycle may be utilized to determine when to avoid insemination or intimate relations to avoid conception. In this manner, the vaginal sensor device may also permit additional conception planning in knowing when to avoid intimate activities so as to serve as a contraception plan, as well as when to pursue intimate activities to facilitate conception.

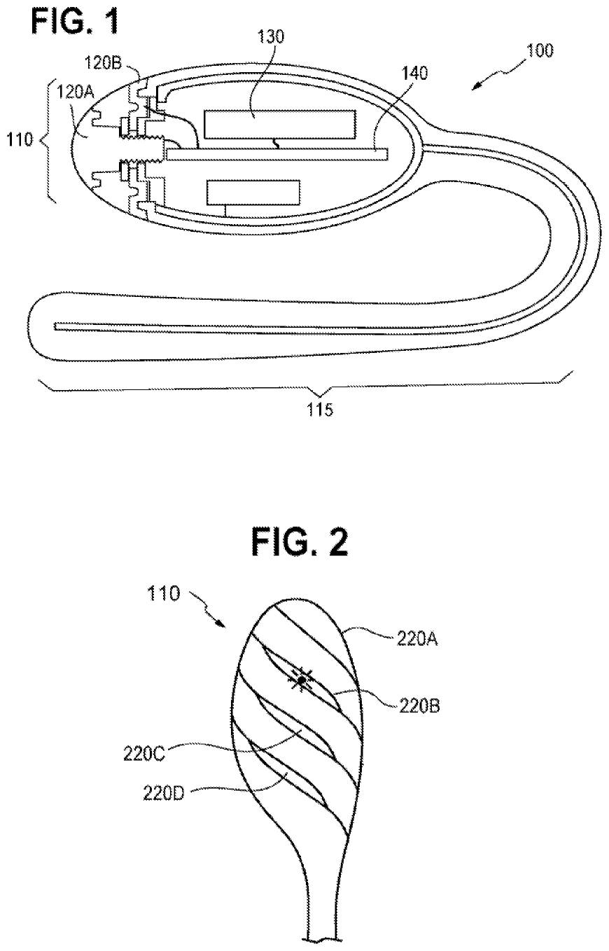

Turning now to FIG. 1, the figure shows a cutaway view of the vaginal sensor device consistent with certain embodiments of the present invention. The vaginal sensor device 100 may comprises a body 110 physically connected to a tail 115. In a non-limiting example, the body 110 may be composed of a flexible material such as a silicon-based compound, however, other flexible compounds having similar properties may be used to manufacture the vaginal sensor device 100 without deviating from the functionality of the vaginal sensor device 100.

The vaginal device 100 may have a first electrode 120A and a second electrode 120B. In a non-limiting example, the first electrode 120A may be positioned at or near an end of the body 110 and the second electrode 120B may be positioned at or near an end of the body 110. The vaginal sensor device 100 may have a power source 130 contained within the body of the vaginal sensor device 110 and operably connected to each of said first and second electrodes. In a non-limiting example, the power source 130 may consist of a battery, such as a lithium-ion battery or other battery that meets the size and power requirements to be contained within the body of the vaginal sensor device 100 and provide the required power for optimal operation of the first and second sensors.

The vaginal sensor device may further comprise an electrical system 140 that may be operably connected to the first electrode 120A and second electrode 120B and the power source 130. The electrical system 140 may be configured to control the operation of the vaginal sensor device 100 through a safe, reliable electrical path installed within the body of the vaginal sensor device body 110 and tail 115. One of more of the first electrode 120A and second electrode 120B may be configured to function as a charge transfer mechanism whenever one or more of a sufficient voltage and a sufficient current is induced on these contacts. The electrical system 140 may be configured to switch one or more of the first electrode 120A and second electrode 120B between charging and measuring functions. In operation, one or more of the first electrode 120A and second electrode 120B may be configured to simultaneously execute charging and measuring to collect measurements from the user.

In a non-limiting example, the electrodes 120A-120B are configured such that upon entry into the vagina of a user they will be in partial contact, and preferably in full contact, with vaginal fluids. To assist with improving contact with vaginal fluids, the vaginal sensor device 100 may be squeezable by the user, where squeezing the device may increase the surface area of the vaginal sensor device 100 that is in contact with the vaginal fluids, including the cervical mucus, of the user.

Placement of the electrodes 120A and 120B at or near the end of the body 110 makes possible measurements with the least variation in results. In this non-limiting example, one or more of the first electrode 120A and the second electrode 120B may be configured to measure a fertility-related parameter. The fertility-related parameter may consist of one or more of electrical impedance, basal body temperature vaginal pH, heart rate on other fertility-related parameters relating to a user's cervical mucus. In a non-limiting example, the vaginal sensor device 100 may measure the impedance of the cervical mucus (in Ohms) of the user. The vaginal sensor device 100 may analyze the measurements of any combination of the collected sensor measurements to characterize and predict a fertility period of the user within the user's monthly fertility cycle.

In an embodiment, the vaginal sensor device 100 may also comprise a communication system 160 that utilizes Near Field Communication (NFC) protocols, such as, in non-limiting examples, Bluetooth, BLE, Zigbee, or other NFC protocols, to communicate collected sensor measurements and receive command and control signals to one or more data processors external to the vaginal sensor device 100. The vaginal sensor device 100 may connect to an external database 150 comprising an electronic data storage element within any of said one or more data processors. The external database 150 may be operable to receive collected sensor measurements and other data and transmit command, control, and other data to the vaginal sensor device 100 to optimize the operation of the system. In a non-limiting example, collected data and sensor measurements may be transmitted to an external data processor, such as a data processor maintained within a cloud computing environment, to permit more in-depth and precise analysis of the collected information than internal analysis by the vaginal sensor device 100. The results from both the analysis performed within the vaginal sensor device 100 and the data processor within the cloud computing environment may be transmitted to a smart device associated with the user to permit the user to view the data analysis and predictions prepared by the system. The user may then utilize the data analysis and predictions to better schedule actions, such as intimate relations, that may produce an optimized probability for conception. Turning now to FIG. 2, the figure shows a cutaway schematic view of the electrodes configured within the vaginal sensor device consistent with certain embodiments of the present invention. The vaginal sensor device body 110 may comprise a plurality of electrodes, these electrodes being designated 220A, 220B, 220C, and 220D. The electrodes 220A-220D are configured to measure diverse fertility-related parameters of a user's body. In a non-limiting example, the electrodes 220A-220D are configured to measure diverse fertility-related parameters relating to the user's vaginal mucus including the cervical mucus. The fertility-related parameters may comprise any of electrical impedance, basal body temperature, vaginal pH, heart rate, and impedance of the cervical mucus and other fertility-related parameters relating to the user's vaginal and cervical mucus.

As shown, the first electrode 220A may comprise a generally cap-like structure that is attached to an edge of the vaginal sensor device body 110. The second electrode 220B may comprise a ring-like electrode that is diagonally attached to a surface of the vaginal sensor device body 110. The third electrode 220C may comprise a ring-like electrode that is diagonally attached to a surface of the vaginal sensor device body 110. The fourth electrode 220D comprises a ring-like electrode that is diagonally attached to a surface of the body vaginal sensor device body 110. In an embodiment, the electrodes 220A-220D may be made of any material known in the art that allows measurement of electrical impedance and is a biocompatible material. Such materials may include, but are not limited to, metal, stainless steel, gold, and any biocompatible material that may allow safe contact of the vaginal sensor device with the body and vaginal tissue of a user. In a non-limiting example, the electrodes 220A-220D are configured such that upon entry into the vagina of a user they will be in partial contact, and preferably in full contact, with vaginal fluids, as well as vaginal and cervical mucus tissue. To assist with improving contact with vaginal fluids, the vaginal sensor device 100 may be squeezable by the user, where squeezing the device may increase the surface area of the vaginal sensor device 100 that is in contact with the vaginal fluids, including the vaginal and cervical mucus, of the user.

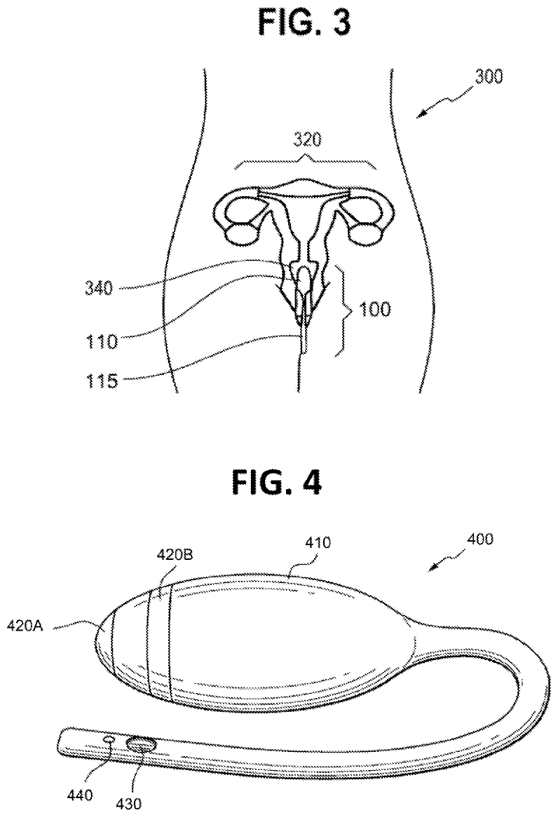

Turning now to FIG. 3, the figure shows a cutaway view of the vaginal sensor device inserted into the vagina of a human female body consistent with certain embodiments of the present invention. This figure illustrates, according to an exemplary embodiment, a front view of a user's human female body 300 showing the female reproductive organs 320 and the vaginal sensor device 100 as inserted in the vagina 350. As depicted, the vaginal sensor device 100 comprises a head 110 and a tail 115. As can be seen, the head 110 of the vaginal sensor device 100 is inserted into the vagina 350, while the tail 115 protrudes from the opening of the vagina 350 in a manner that allows holding of the tail 115 and removal of the vaginal sensor device 100 out of the vagina 350.

Turning now to FIG. 4, the figure shows a side view of the vaginal sensor device having a tail in usable configuration consistent with certain embodiments of the present invention. As shown the vaginal sensor device 100 comprises at least a first electrode 120A and a second electrode 120B, where the first electrode 120A comprises a generally cap-like structure that is attached to an edge of the body 110. As depicted, the second electrode 120B comprises a ring-like electrode that is horizontally attached to a surface of the body 110. In a non-limiting example, one or more of the first electrode 120A and the second electrode 120B may comprise a screw.

The vaginal sensor device 100 further comprises an on/off button 410. As depicted, the on/off button 410 is positioned on the tail. Preferably, although not necessarily, the on/off button 410 is positioned on a part of the tail that is not inserted into the vagina. As depicted, the on/off button 410 is positioned on a distal side of the tail relative to the body 110. The on/off button 410 is configured to be pressable or movable by a user to do one or more of turn on the vaginal device 100 and turn off the vaginal sensor device 100.

In an embodiment, the vaginal device 100 further comprises an optional light signal 420. In a non-limiting example, the light signal 420 is positioned on the tail. Preferably, although not necessarily, the light signal 420 is positioned on a part of the tail that is not inserted into the vagina. The light signal 420 may be positioned on a distal side of the tail relative to the body 110. The light signal 420 may be configured to indicate whether the vaginal device 100 is turned on or off In a non-limiting example, the light signal 420 may comprise a light source emitting colored light indicating the on/off state of the vaginal device 100. In this non-limiting example, the light signal 420 may emit green light when the vaginal device 100 is turned on, and may emit yellow light when the vaginal device 100 is turned off.

In an embodiment, the electrodes 120A-120B may each be physically continuous. At least one of the electrodes 120A-120B may comprise a plurality of electrode dots closely attached on the surface of the body 110, forming patterns on the surface of the body 110, for example comprising stripes similar to the ones depicted.

As previously described, the electrodes 120A-120B may be configured such that upon entry into the vagina of a user they will be in partial contact, and preferably in full contact, with vaginal fluids. To assist with improving contact with vaginal fluids, the vaginal sensor device 100 may be squeezable by the user, where squeezing the device may increase the surface area of the vaginal sensor device 100 that is in contact with the vaginal fluids, including the cervical mucus, of the user.

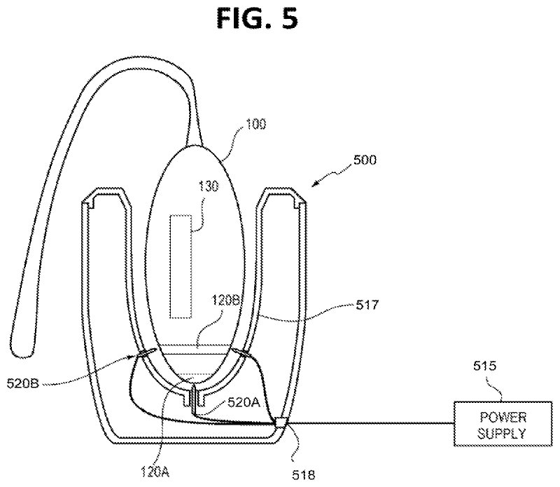

Turning now to FIG. 5, the figure shows a detail view of the vaginal sensor device and recharging cradle consistent with certain embodiments of the present invention. In an embodiment, the vaginal sensor device 100 comprises the first electrode 120A, the second electrode 120B, and the power source 130. The system 500 comprises a vaginal sensor device 100 and a charging cradle 510 configured to do one or more of protect the vaginal sensor device 100 and hold the vaginal device 100 in place for charging. The charging cradle 510 may be operably connected to an external power supply 515. The charging cradle 510 may further comprise a charging interface 517 configured to electrically connect with and charge at least one of the electrodes 120A-120B. The charging cradle 510 is configured to charge the power source 130 of the vaginal sensor device 100 through the charging interface 517. The charging cradle 510 further comprises a power port 518. The power port 518 may be configured to receive power from the external power supply 515.

In a non-limiting example, the external power supply 515 comprises a direct current (DC) power supply or a rectified AC power supply when a charging cord and AC/DC rectification element are supplied (not shown). In an embodiment, the charging interface 517 comprises three charging nodes 520A, 520B and 520C. The three charging nodes 520A-520C are configured to electrically connect with corresponding electrodes 120A-120B. As depicted, the first charging node 520A is configured to electrically connect with the first electrode 120A. The second charging node 520B and the third charging node 520C are both configured to electrically connect with the second electrode 120B. In this non-limiting example, one or more of the charging nodes 520A-520C is spring loaded.

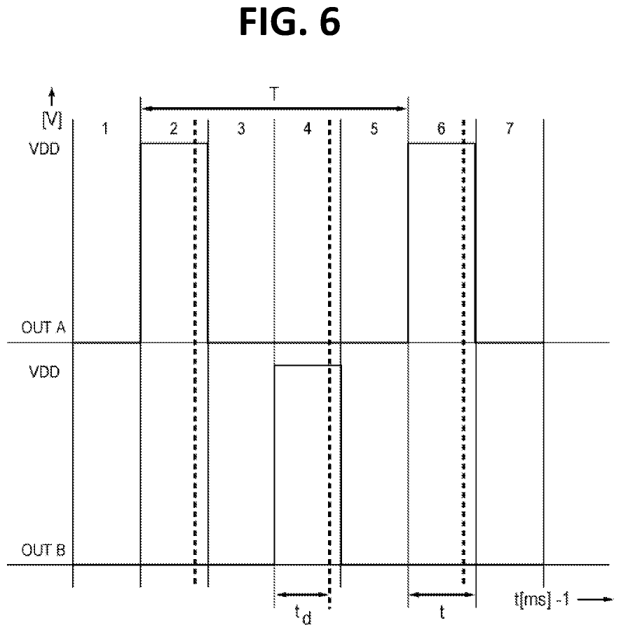

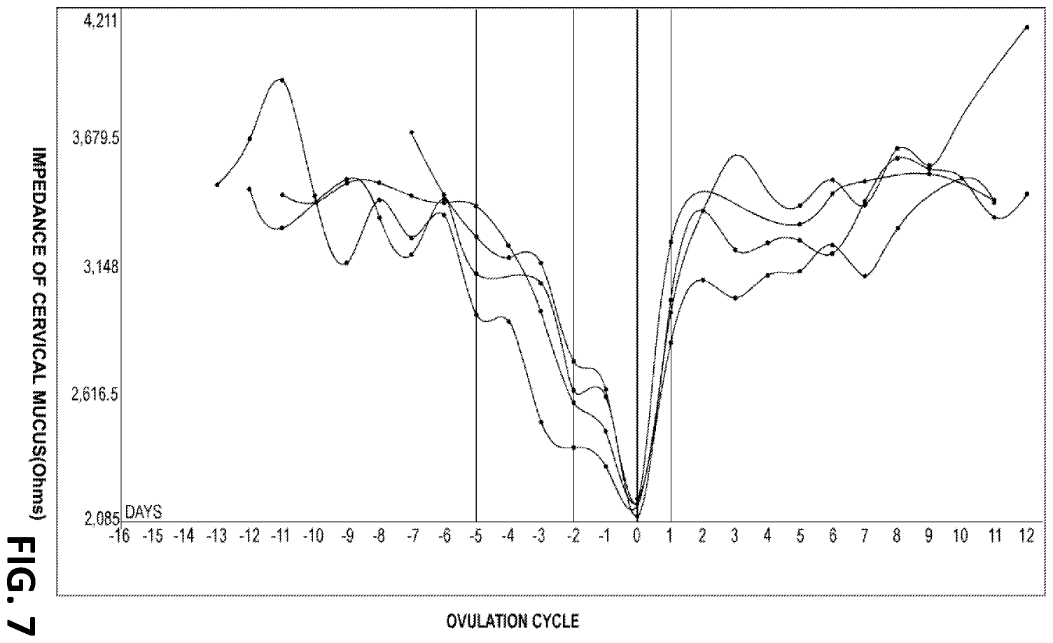

Turning now to FIG. 6, the figure shows a detail view of the voltage over time graph created during measurement activities of the vaginal sensor device consistent with certain embodiments of the present invention. In an embodiment, this figure presents a graph showing representative measurements made by the vaginal device of voltage (regulated voltage or VDD, in volts) over time (t). T indicates the time period of repetition of measurements; t indicates voltage pulse width, namely the length of voltage pulse, and to indicates sampling delay, namely the time between pulse start and sampling time.

In an embodiment, the vaginal sensor device measures bipolar resistivity of vaginal mucus. This is achieved by using alternating voltage pulses, which are led to electrodes via a resistor that is limiting the current. At the same time the resistor is limiting the current, it is also measuring the current. Using the collected measurement data, the vaginal sensor device may calculate the electrical impedance of the cervical mucus. In a non-limiting example, accuracy in the measurement data for the impedance of the user's cervical mucus may be improved by use of multiple electrodes for one or more of resistive tomography and impedance tomography.

Turning now to FIG. 7, the figure presents a graph of cervical impedance vs. day of a user ovulation cycle collected by the vaginal sensor device consistent with certain embodiments of the present invention. In an embodiment, the system may be configured to predict menstrual cycles and more particularly predict ovulation and fertile period based upon an analysis of the collected sensor data. The analysis may be performed quickly within the processor associated with a mobile device such as, in non-limiting examples, a smart phone, iPad, tablet computer, or other portable computing device. Additionally, the collected data may be transmitted to one or more data processors operational within a cloud environment to accept the collected data and perform a more accurate and optimized analysis within the more computationally powerful cloud data processors. In an embodiment, there is provided a dedicated application available for download and installation on a mobile device such as smartphone, iPad, tablet, smart watch, or other mobile device having an integral data processor. Once the application is installed in the mobile device, the user may be prompted to register with the analysis system to be included within a user database. The user may then be prompted to pair the application with the vaginal sensor device. The vaginal sensor device may be configured to start automatically every time it is taken out from a charging station or every time it is pressed by a user.

In an embodiment, every morning a user is prompted to turn on the vaginal device and to insert the vaginal device into the vagina. The user may then be prompted to start the application and open a device tab within the application. At this stage, the vaginal sensor device may be configured to synchronize automatically with the application. If synchronization does not happen, the user is prompted to make sure that an NFC communication line between the smartphone and the vaginal device, for example Bluetooth, BLE, or Zigbee, is turned on. Alternatively, the user may manually synchronize the vaginal device with the application by selecting a ‘SYNC’ button located in a device tab of the application. Upon insertion of the vaginal sensor device within the vagina, the vaginal sensor device is configured to begin measuring fertility-related parameters. In a non-limiting example, the synchronization should preferably require approximately 5 minutes to complete.

In this embodiment, the vaginal sensor system may collect a user's measured body data such as basal body temperature, vaginal pH—for example based on impedance of the vaginal fluids, heart rate and similar body data measurements. The data are preferably collected every morning by inserting the vaginal sensor device into the vagina for a certain period of time that is enough to collect the necessary data, for example substantially 5 minutes. The collected measurement sensor data are transferred to a mobile device, for example a smartphone, iPad, tablet, smart watch, or other portable processor. The mobile device may run a computer program, namely an application that processes the data to provide a quick analysis and prediction of a fertility window for the user. Simultaneously the vaginal sensor device may submit the collected measurement data over the NFC communication channel to a data processor within a cloud environment. The cloud-based data processor may perform a more detailed and optimized analysis and prepare an analysis and prediction or recommendation. The cloud-based data processor may transmit to the mobile device the results of the analysis, where the mobile device receives the processed data from the cloud and displays the data to the user via a display associated with the mobile device of whatever type. Thus, the analysis application may display the user's measurements, cycles, last period and most importantly a prediction of one or more of a next fertility period, ovulation time, and a next period.

In an embodiment, during the measurement of the fertility-related parameters, the user may see the status of measurement in the ‘Device Tab’ of the application. Once the measurements have completed and the data values have been collected, the user may be prompted to the remove the vaginal device from the vagina.

Upon removal of the vaginal sensor device from the vagina, the device may be cleaned, for example, with water and soap and then dried. Then the vaginal sensor device may be returned to a charging dock for charging the power source of the vaginal sensor device until next time of usage. According to one embodiment, charging may take approximately 30 minutes.

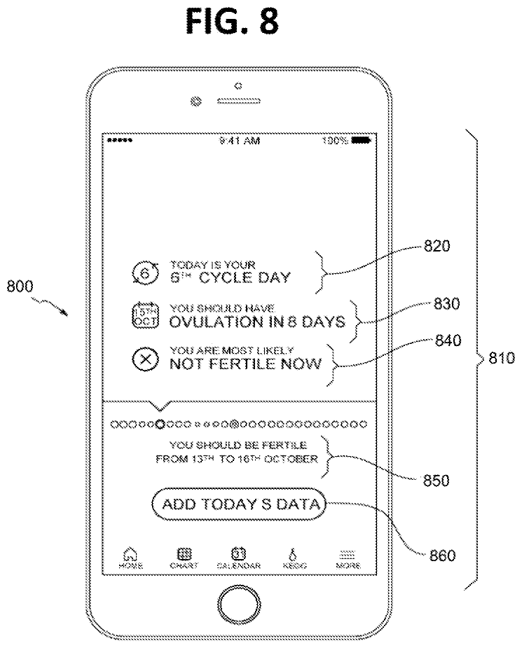

Turning now to FIG. 8, the figure shows an exemplary screenshot 800 of a home page view 810 of an operation application configured to be used with the vaginal sensor device consistent with certain embodiments of the present invention.

In an embodiment, the vaginal sensor device may be connected to an analytical and reporting application that may be downloaded to a mobile device, such as, in non-limiting examples, a smart phone, smart watch, iPad, tablet, laptop, or other mobile device having a data processor. Upon download, when the application is used for the first time the user may be prompted to create a profile in which to store the collected sensor measurement data and user entered data that is associated with the user. The user entered data may include personal and demographic information about the user such as information about the user's last period, regularity of the user's period cycle, age, and other demographic information. The information entered by the user may be used as input to the analysis algorithm to optimize the predictions and recommendations for the user, to customize the results to the user. The collected sensor measurement data and user entered data may be stored within an electronic storage media within the mobile device and may also be communicated from the mobile device to a cloud-based data processor through an established network communication channel such as a Wi-Fi or other data connection.

The home page view 810 provides information on the display of the mobile device that displays analytical data and prediction data to the user based upon both collected sensor data and data input by the user. In this figure the display may present as initial information on the home page view 810 the particular day of the user's current cycle 820, an ovulation prediction 830, a current fertility window 840, a next or proximal fertility window 850, and an input selection area 860.

In an embodiment, the cycle day 820 represent a number of days from the user's most recent period. In a non-limiting example, as shown in FIG. 8 the user is informed that “Today is your 6^{th} cycle day.” This information permits the user to remain informed about the cycle day without losing track.

In this embodiment, the ovulation prediction 830 may comprise a prediction of one or more of a next ovulation within the current cycle. In a non-limiting example, as shown the ovulation prediction comprises the text, “You should have an ovulation in 8 days” permitting the user to understand the approximate time when they have the greatest likelihood of an ovulation.

In this embodiment, the current fertility window 840 comprises a prediction as to the user's greatest probability of fertility within the current cycle. In this non-limiting example, as shown the fertility window comprises the text “You are most likely not fertile now.”

In this embodiment, the next fertility window 850 comprises a prediction of the fertility window of the user. The fertility window 850, as previously described, comprises a pre-determined period of when to time insemination to have the greatest likelihood of impregnation. In this non-limiting example, the fertility window may be set as a four-day period and the fertility window prediction comprises the text “You should be fertile from the 13^{th} through the 16^{th} of October”, once again presenting the user with a customized and optimized prediction of a fertility window to permit the user to plan for an insemination event based upon this prediction.

In this embodiment, the input selection area 860 may be selected by the user to input any information that may assist in the analysis and prediction of the fertility window for a particular user. When selected, the user may be transferred to an input window screen to permit the user to interact with the application and input any desired information utilizing the mobile device.

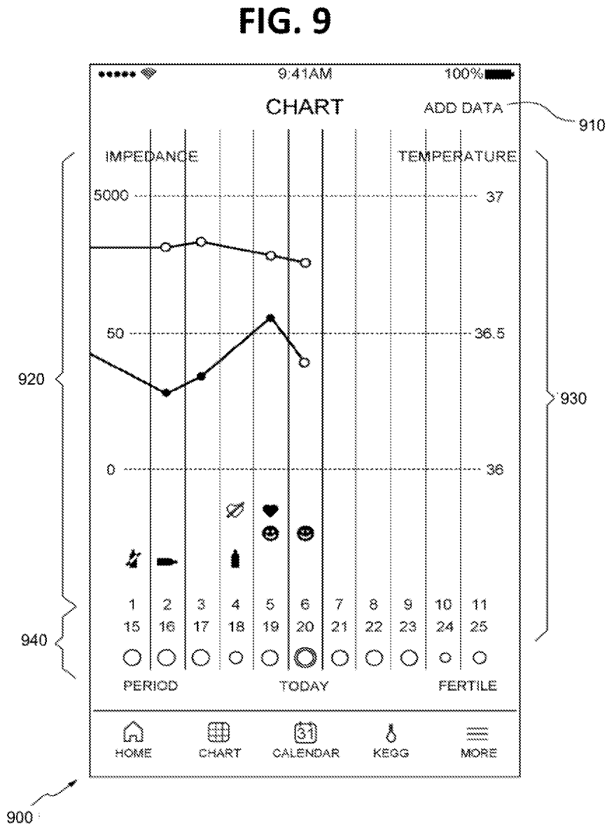

Turning now to FIG. 9, the figure shows an in-use view of the chart page of the operation application in use with the vaginal sensor device consistent with certain embodiments of the present invention. In this embodiment, included on the chart page 900 may be a data addition button 910, an impedance record 920 showing measured impedance of the user's cervical mucus, a temperature record 930 showing measured basal body temperatures of the user in Celsius, and a thumbnail calendar 940. In a non-limiting example, if the user presses the data addition button 910, the user may be presented with a data addition page and is invited to input data into the mobile device.

In this embodiment, the user has the option to select Fahrenheit instead of

Celsius for the temperature record 930. The thumbnail calendar 940 may display the user's current fertility cycle, showing the current day of the fertility cycle for the user as day 20. The thumbnail calendar 940 comprises a fertile period indicator. If the user clicks on the thumbnail calendar 940, the user may be presented with the calendar page display.

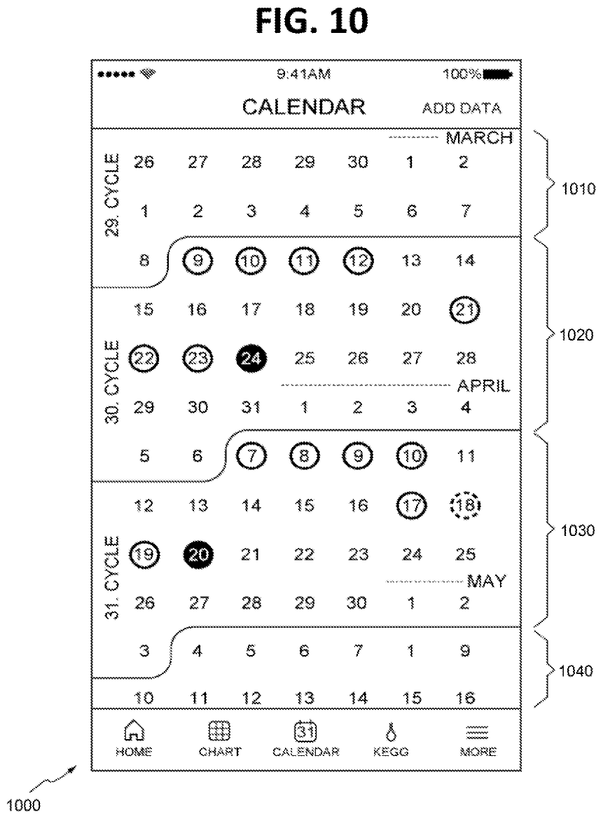

Turning now to FIG. 10, the figure shows an in-use view of the calendar page of the operation application in use with the vaginal sensor device consistent with certain embodiments of the present invention. In an embodiment, graphical visualizations of parts of four user fertility cycles are presented, a first fertility cycle 1010 for which only the latter 15 days are shown, a second fertility cycle 1020 shown in full, a third fertility cycle 1030 also shown in full, and a fourth fertility cycle 1040, for which only the first 13 days are shown. Days of each cycle are marked as appropriate as one or more of a fertile day, an infertile day, a day of ovulation, a day of a period, and a likely day to conceive. Both past fertility cycles and projected future fertility cycles can be viewed by the user using the calendar page 1000.

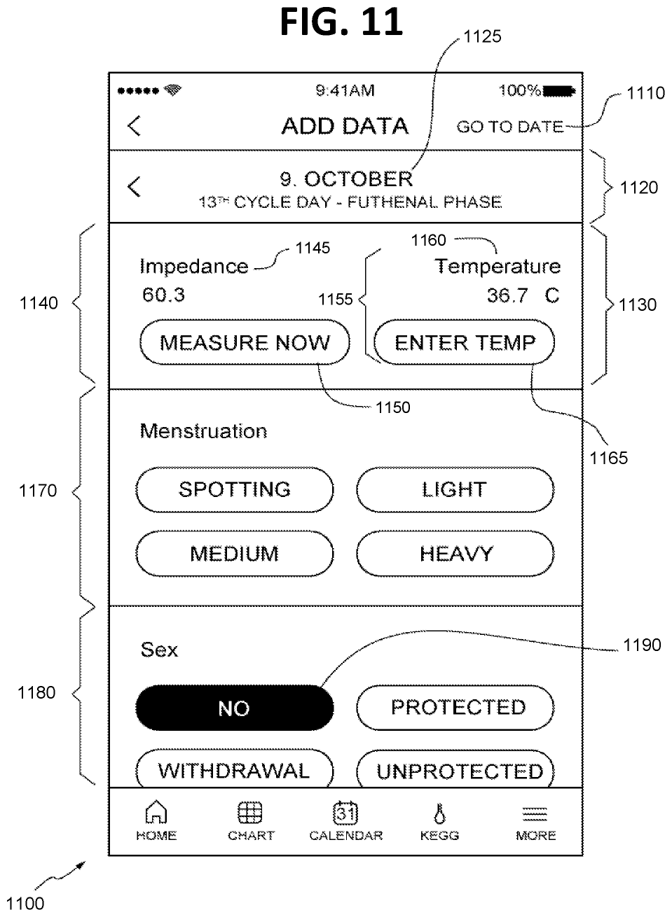

Turning now to FIG. 11, the figure shows an in-use view of the data addition page 1100 of the operation application in use with the vaginal sensor device consistent with certain embodiments of the present invention. In an embodiment, the data addition page 1100 may prompt the user to provide additional data through manual input, for example, parameters like: mood, drinking, manual temperature measurements, sex, menstruation data, and the like. The input of these data is optional, but the addition of more data provides for better optimization and confidence of the fertility prediction information supplied to the user.

In this embodiment, the data addition page 1100 may comprise a “go to date” button 1110. If the user selects this “go to date” button 1110, the user may go to any desired date of interest to input additional data. The data addition page 1100 further comprises a date window 1120 displaying a date of interest 1125, and also listing the day of the user's fertility cycle and the phase of the user's fertility cycle. In a non-limiting example, the date of interest 1125 shows a date of October 9. By default, the date window 1120 displays as the date of interest 1125 the current date and information on the current date.

In this embodiment, the impedance and temperature window 1130 comprises a vaginal impedance window 1440 comprising an impedance value window 1145 showing the user's cervical mucus impedance value on the date of interest 1125. In a non-limiting example, the impedance value window 1145 shows the user's cervical impedance value on October 9 as 60.3. The cervical impedance window 1440 further comprises an impedance measurement button 1450 which, upon selection, allows the user to manually collect an additional measurement of the cervical mucus impedance in real time to be added to the previously collected data values. Additionally, the impedance and temperature window 1130 further comprises a temperature window 1155 comprising a temperature value window 1160 showing the user's basal body temperature on the date of interest 1125. In a non-limiting example, the temperature value window 1155 displays the user's basal body temperature on October 9 as 36.7 degrees Celsius. The user has the option of selecting Fahrenheit instead of Celsius for displaying the temperature value in the temperature window 1155. The temperature window 1155 further comprises a temperature measurement button 1165 which, upon selection, allows the user to manually collect an additional measurement of the user's basal body temperature in real time to be added to the previously collected data values.

The data addition page 1100 further comprises a menstruation window 1170 that allows the user to choose a menstruation level as one or more of spotting, light, medium, and heavy. The user's selection is then indicated by an appropriate darkened bubble. In a non-limiting example, the user has not selected a menstruation level.

The data addition page 1100 further comprises a sex window 1180 that allows the user to choose a sexual activity level as one or more of no sexual activity, protected sex, withdrawal sex, and unprotected sex. The user's selection is then indicated by an appropriate darkened bubble. In a non-limiting example, the darkened “no sexual activity” button 1190, indicates that the user has selected a sexual activity level of no sexual activity.

Although not shown, a “Your Profile” page may display current selected and stored user personal information to the user. Using buttons on this page, the user may modify profile information provided during registration for the application and/or subsequently to registration.

Although not shown, an “App Lock” page provides the user an opportunity to secure the application and the data with a personal identification number (PIN) code, so no one may be able to access the application except the user or those to whom the user has provided the PIN information.

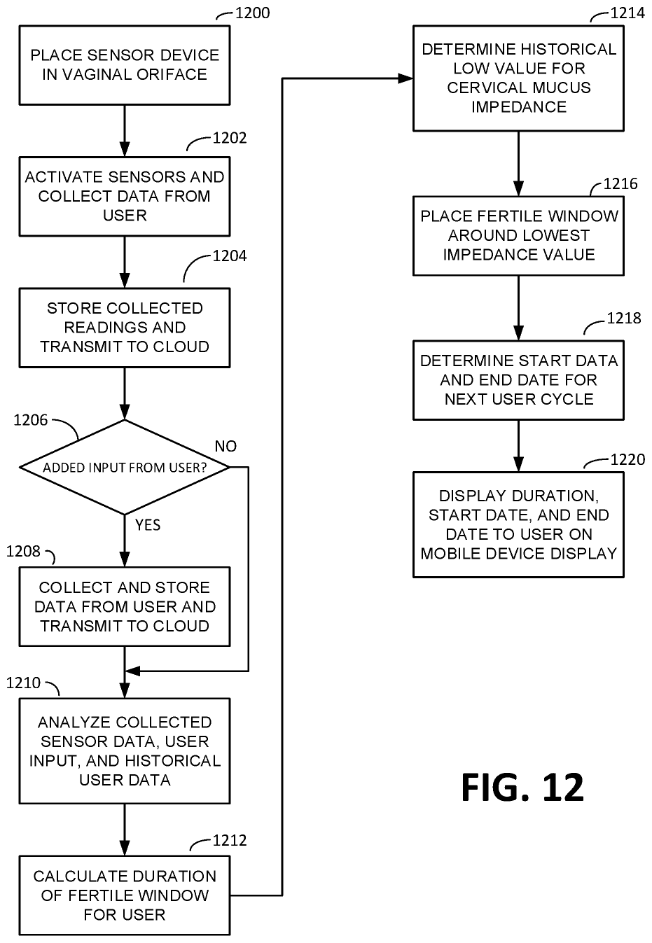

Turning now to FIG. 12, the figure is a flow diagram for the use of the vaginal sensor device to predict and characterize a user fertility window consistent with certain embodiments of the present invention.

In an embodiment, at 1200 to begin the prediction and characterization of the user fertility window the user may place the vaginal sensor device within the vaginal orifice. At 1202 an alternating signal is input to the vaginal sensor device to activate the sensors. The sensors are active to collect one or more measurements of electrical impedance, basal body temperature, vaginal pH, heart rate, and other fertility-related parameters relating to a user's vaginal mucus. In a non-limiting example, the vaginal sensor device 100 may measure the impedance of the cervical mucus (in Ohms) of the user. At 1204 all of the collected measurement data is stored within an electronic storage medium both within a mobile device associated with the user and in a cloud-based electronic storage medium. The mobile device electronic storage medium may be associated with the user's mobile device connected to the vaginal sensor device through an NFC protocol such as Bluetooth, BLE, or other near field communication capability. At 1206 the user may decide or be required to add information that is specific to the user such as cycle, menstruation, intimacy frequency, demographic information, or other information that is important to the user's cycle. If the user wishes to add information, the user may perform data input within the mobile application downloaded to the mobile device. At 1208 this input date from the user may be captured and stored both within the mobile device and transmitted for storage to the cloud-based electronic storage medium.

When the user has completed any data input or has decided not to input any additional information, at 1210 an analytical algorithm utilizes the collected sensor measurements, user input data, and historical user data either within the application in the mobile device and/or within a cloud-based data processor. At 1212, the analytical algorithm may calculate the duration of a fertile window that is customized for the user. At 1214, the analytical algorithm may be active to determine the historical low value of the cervical mucus impedance for the user and the day within the user's cycle when that low value is recorded on a historical basis. At 1216, the analytical algorithm places the calculated fertile window duration, in days, around the historical low cervical mucus impedance day within the user's cycle such that the user is provided with at least a 2-3 day time span prior to the historical low cervical mucus impedance day. At 1218, the analytical algorithm determines the start data and end data as dates on a calendar that are determined from the historical day within a user's cycle and provides the fertility window duration, start date, end date and low cervical mucus impedance values as a prediction to be displayed to the user on the display of the mobile device at 1220.

While certain illustrative embodiments have been described, it is evident that many alternatives, modifications, permutations and variations will become apparent to those skilled in the art in light of the foregoing description.

What is claimed is:

1. A system for determining a fertility window, comprising:
a vaginal sensor device;
said vaginal sensor device configured to collect measurements of a user's fertility related parameters for a current user cycle;
storing said collected measurements within said vaginal sensor device;
transmitting said collected measurements to a data processor within a cloud server and storing said collected measurements within an electronic storage file associated with said user;
said data processor analyzing said collected measurements, historical data, and current data input from said user to calculate a fertility window for said current user cycle;
said data processor determining a fertility window start date and end date for said current user cycle;
displaying said start date and said end date to said user on a display accessible to said user;
said user instituting one or more conception activities within said fertility window start date and end date.

2. The system of claim 1, where the collected measurements comprise any of measurements of electrical impedance, basal body temperature, vaginal pH, impedance of cervical mucus, and other fertility-related parameters relating to a user's vaginal mucus.

3. The system of claim 1, where the vaginal sensor device is in near field communication with a mobile device.

4. The system of claim 1, where said mobile device comprises any of a laptop, smart phone, smart watch, iPad, tablet, or other mobile device having a near field communication capability.

5. The system of claim 1, where current input data from a user comprises any of a user's information related to the user's cycle, menstruation, intimacy frequency, demographic information, or other information that is important to the user's current cycle.

6. The system of claim 1, where said historical data comprises any of a user's information related to the user's cycle, menstruation, intimacy frequency, demographic information, or other information for past cycles and stored within said electronic storage file maintained within said cloud server for said user.

7. The system of claim 1, comprising said data processor calculating the duration of a fertility window for said current user cycle.

8. The system of claim 7, further comprising determining said start date and end date for said fertility window from said duration of the fertility window for said current user cycle.

9. The system of claim 1, further comprising determining the historical low value of the cervical mucus impedance for the user's current cycle and establishing a 2 to 3 day time span prior to the historical low cervical mucus impedance day.

10. The system of claim 1, further comprising displaying the fertility window duration, start date, end date, and low cervical mucus impedance values as predictions for said current user cycle.

11. A method for determining a fertility window, comprising:
collecting measurements of a user's fertility related parameters for a current user cycle;
storing said collected measurements within a local electronic storage medium;
transmitting said collected measurements to a data processor within a cloud server and storing said collected measurements within an electronic storage file associated with said user;
analyzing said collected measurements, historical data, and current data input from said user to calculate a fertility window for said current user cycle;
determining a fertility window start date and end date for said current user cycle;
displaying said start date and said end date to said user on a display accessible to said user;
said user instituting one or more conception activities within said fertility window start date and end date.

12. The method of claim 11, where the collected measurements comprise any of measurements of electrical impedance, basal body temperature, vaginal pH, impedance of cervical mucus, and other fertility-related parameters relating to a user's vaginal mucus.

13. The method of claim 11, where the vaginal sensor device is in near field communication with a mobile device.

14. The method of claim 11, where said mobile device comprises any of a laptop, smart phone, smart watch, iPad, tablet, or other mobile device having a near field communication capability.

15. The method of claim 11, where current input data from a user comprises any of a user's information related to the user's cycle, menstruation, intimacy frequency, demographic information, or other information that is important to the user's current cycle.

16. The method of claim 11, where said historical data comprises any of a user's information related to the user's cycle, menstruation, intimacy frequency, demographic information, or other information for past cycles and stored within said electronic storage file maintained within said cloud server for said user.

17. The method of claim 11, comprising said data processor calculating the duration of a fertility window for said current user cycle.

18. The method of claim 17, further comprising determining said start date and end date for said fertility window from said duration of the fertility window for said current user cycle.

19. The method of claim 11, further comprising determining the historical low value of the cervical mucus impedance for the user's current cycle and establishing a 2 to 3 day time span prior to the historical low cervical mucus impedance day.

20. The method of claim 11, further comprising displaying the fertility window duration, start date, end date, and low cervical mucus impedance values as predictions for said current user cycle.
